(19) United States
(12) Patent Application Publication
(10) Pub. No.: US 2007/0233084 A1
(43) Pub. Date: Oct. 4, 2007

(54) IMPLANTABLE SPINOUS PROCESS PROSTHETIC DEVICES, INCLUDING CUFFS, AND METHODS OF FABRICATING SAME

(75) Inventors: Randal R. Betz, Ocean City, NJ (US); Edward Miller, Cincinnati, OH (US); Rebeccah Brown, Decatur, GA (US); Guilhem Denoziere, Atlanta, GA (US)

Correspondence Address: MYERS BIGEL SIBLEY & SAJOVEC, PO BOX 37428, RALEIGH, NC, 27627, US

(73) Assignee: SpineMedica Corporation

(21) Appl. No.: 11/625,845

(22) Filed: Jan. 23, 2007

Related U.S. Application Data
(60) Provisional application No. 60/761,882, filed on Jan. 25, 2006.

(51) Int. Cl. A61F 2/30

(52) U.S. Cl. 606/61

(57) ABSTRACT

Spinous process implants have an elastomeric cuff and/or integral elatomeric spacer with the cuff sized and configured to encase at least a minor portion of a first spinous process.

### RELATED APPLICATIONS

This application claims priority to U.S. Provisional Application Ser. No. 60/761,882, filed Jan. 25, 2006, the contents of which are hereby incorporated herein by reference as if recited in full herein.

### FIELD OF THE INVENTION

The invention relates to spinal implants.

### BACKGROUND OF THE INVENTION

The spinous process is the portion of a vertebra that protrudes posteriorly from the spinal column. The spinous process is the most posterior extension of the spine. The spinous processes provide the “bumps” on the midline of the back.

In the past, several treatments have been proposed for back pain, injury and degenerative conditions, such as spinal fixation or fusing adjacent spinous processes, which typically inhibit motion. Others have proposed implanting a spacer that is placed between two spinous processes of two vertebrae. Examples of the latter include devices described in U.S. Patent Application Publication Nos. 20050261768 and 20040106995.

Despite the above, there remains a need for alternative treatment options for a spinous process.

### SUMMARY OF EMBODIMENTS OF THE INVENTION

Embodiments of the invention are directed to spinal process implants.

Some embodiments are directed to spinous process implants that include an elastomeric cuff sized and configured to encase at least a minor portion of a first spinous process.

Other embodiments are directed to methods of fabricating a spinous process implant. The methods include molding elastomeric material into a unitary body having a substantially tubular shape with a spinous process receiving cavity therein.

The molding step can include forming the substantially tubular shape so that one end is closed and one end is open or so that both ends are open.

Some embodiments are directed to spinous process implants that include: (a) a flexible crystalline polyvinylalcohol cryogel body configured to reside between neighboring first and second spinous processes; and (b) at least one bone attachment member extending from the elastomeric body. The bone attachment member, configured to attach to the first spinous process to hold the elastomeric body in position. In position, the implant is configured to allow motion between adjacent spinous process bones.

Other embodiments are directed to spinal process implants that include: (a) a first cuff sized and configured to receive at least a minor portion of a first spinous process therein; and (b) a second cuff attached to the first cuff, the second cuff sized and configured to receive at least a minor portion of a second adjacent spinous process therein, whereby the implant allows motion between the first and second spinous processes.

The implants may include an elastomeric cushion member disposed between the first and second cuffs. In particular embodiments, the elastomeric cushion member is formed of PVA hydrogel.

Still other embodiments are directed to medical kits. The kits include at least one sterilized spinous process cuff enclosed in an aseptic or sterile package.

The cuff in the kit may be configured with a sleeve that merges into an integral elastomeric spacer with the cuff can be configured to encase a posterior portion of the spinous process.

Additional embodiments are directed to methods of treating a spinous process. The methods include sliding an elastomeric cuff onto a spinous process. The method may optionally also include removing a posterior portion of the spinous process before the implanting step.

The method can include attaching tabs extending from the cuff to bone and/or attaching a strap or band to the cuff.

Further features, advantages and details of the present invention will be appreciated by those of ordinary skill in the art from a reading of the figures and the detailed description of the embodiments that follow, such description being merely illustrative of the present invention.

### BRIEF DESCRIPTION OF THE DRAWINGS

FIG. 1 is a perspective view of a spinous process cuff in position on a spinous process according to embodiments of the present invention.

FIG. 2A is a side perspective view of the cuff shown in FIG. 1.

FIG. 2B is a side perspective view of the cuff shown in FIG. 1 with a mesh scaffold according to embodiments of the invention.

FIG. 2C is a cross-sectional view of the cuff with mesh scaffold shown in FIG. 2B according to embodiments of the invention.

FIG. 2D is an enlarged view of an exemplary configuration of exposed mesh of the cuff shown in FIGS. 2B and 2C according to embodiments of the invention.

FIG. 3A is a perspective view of the cuff shown of FIG. 1 with an anchoring band according to embodiments of the present invention.

FIG. 3B is a perspective view of the cuff of FIG. 1 with a bone anchoring tab(s) according to embodiments of the present invention.

FIG. 4 is an enlarged partial section view showing the anchoring tab of FIG. 3B according to embodiments of the present invention.

FIG. 5A is a schematic illustration showing that a cuff may be configured as one or more narrow members according to some embodiments of the present invention.

FIG. 5B is a schematic illustration of a cuff comprising a single narrow member according to embodiments of the present invention.

FIG. 5C is a schematic illustration illustrating the use of a plurality of narrow cuffs according to embodiments of the present invention.

FIGS. 6-8, 9A and 9B are side perspective views of different exemplary cuff configurations according to embodiments of the present invention.

FIG. 10 is a schematic section view of the spine illustrating two cuffs in an exemplary position according to embodiments of the present invention.

FIGS. 11A and 11B are schematic posterior views of a spinal process with exemplary integral double cuff configurations according to embodiments of the present invention.

FIG. 12A is a schematic posterior view of two adjacent processes with an implant in position according to other embodiments of the present invention.

FIG. 12B is a side view of the device shown in FIG. 12A.

FIGS. 13A and 13B are schematic posterior views of two adjacent processes with an implant in position according to further embodiments of the present invention.

FIG. 14A is a side cutaway view of a cuff with an integral flexible spacer portion according to still other embodiments of the present invention.

FIG. 14B is a posterior view of a cuff with an integral spacer such as that shown in FIG. 14A according to embodiments of the present invention.

FIGS. 14C and 14D are posterior views of a cuff with integral spacer according to other embodiments of the present invention.

FIG. 15 is a flow chart of steps that can be used to carry out other embodiments of the present invention.

FIG. 16A is a schematic section view of a mold that can be used to fabricate a molded cuff according to embodiments of the present invention.

FIG. 16B is an end view of the mold shown in FIG. 16A.

FIG. 16C is a side perspective view of a cuff provided by the mold shown in FIG. 16A according to embodiments of the present invention.

FIG. 17A is a schematic section view of a mold that can be used to fabricate a molded cuff according to embodiments of the present invention.

FIG. 17B is a side perspective view of a cuff provided by the mold shown in FIG. 17A according to embodiments of the present invention.

### DETAILED DESCRIPTION

The present invention now is described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity. Broken lines illustrate optional features or operations unless specified otherwise.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention. The sequence of operations (or steps) is not limited to the order presented in the claims or figures unless specifically indicated otherwise.

The term “thin,” means that the device has a thickness that is less than about 6 mm, typically between about 1-5 mm. The term “conformal” means that the implant material or member is sufficiently flexible to conform to a target structure's shape.

The term “cuff” refers to an implant member that, in position, defines a space that can receive at least a minor portion of a length of spinous process bone and encase substantially all (typically cover all) of the circumferential perimeter thereof (including an upper or lower portion thereof). The cuff can have a closed posterior end portion or an open posterior end portion.

The term “mesh” means any flexible material in any form including, for example, knotted, braided, extruded, stamped, knitted, woven or otherwise, and may include a material with a substantially regular foramination pattern and/or irregular foramination patterns. Exemplary pore sizes for tissue ingrowth and/or bone ingrowth into an exposed mesh scaffold that may be used is typically between about 0.5 mm to about 1 mm.

The term “exposed mesh scaffold” refers to mesh that has been processed to inhibit molded implant material from attaching to portions of the mesh or to remove molded material therefrom to thereby expose mesh at least to a partial depth in the mesh thickness to allow for tissue in-growth. That is, the mesh can be moldably attached to the implant and the mold material can be removed or inhibited from entering the mesh at localized regions to provide the exposed mesh scaffold surface to a partial or full depth of the mesh at one or more locations or segments of the mesh attached to the molded implant body.

The term “macropores” refers to apertures having at least about a 0.5 mm diameter or width size, typically a diameter or width that is between about 1 mm to about 3 mm, and more typically a diameter or width that is between about 1 mm to about 1.5 mm (the width dimension referring to non-circular apertures). The macropores may promote bony through-growth for increased fixation and/or stabilization over time.

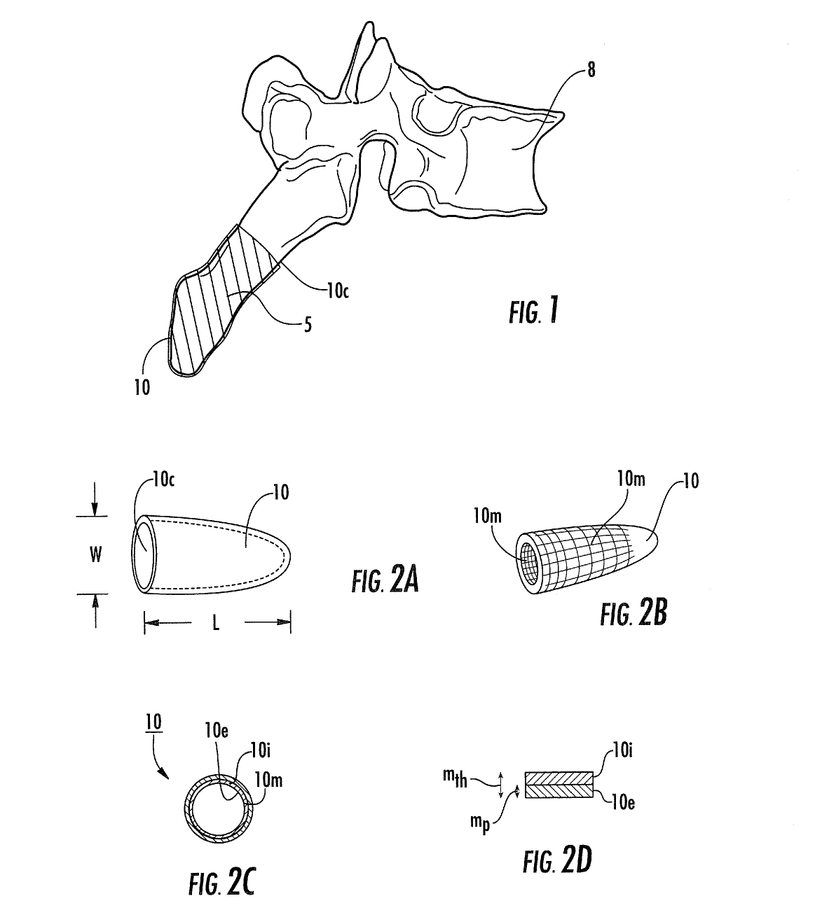

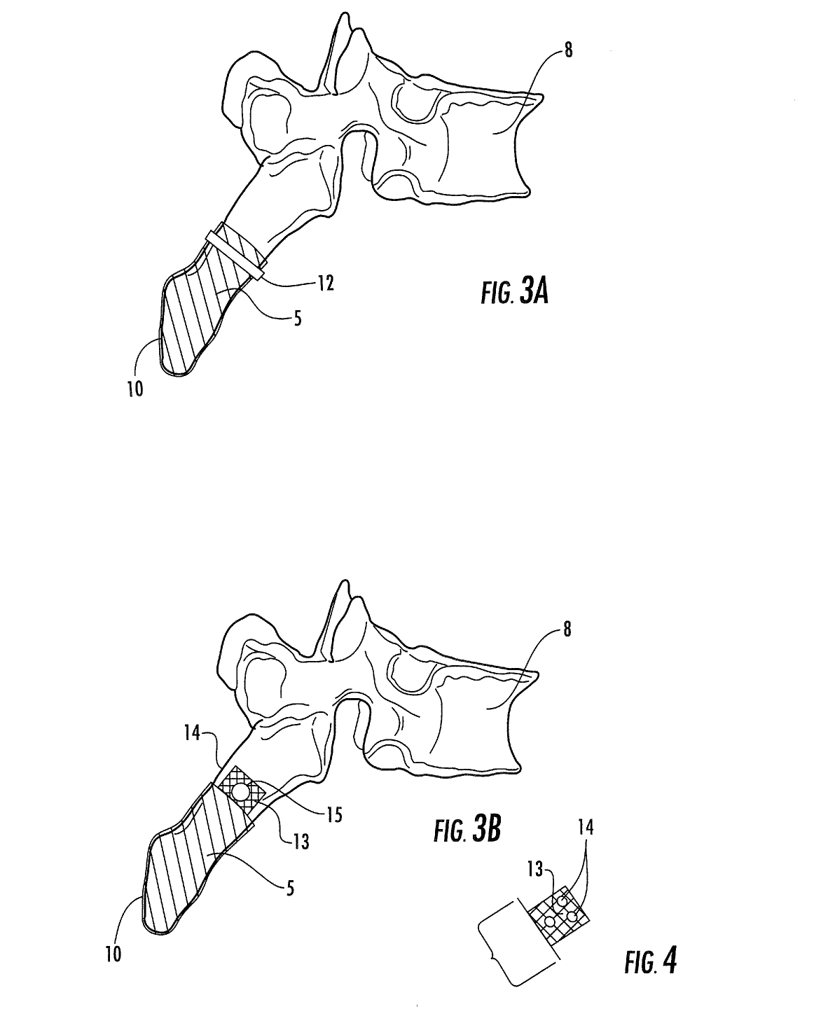

FIG. 1 illustrates a spinous process 5 on a rear side of the spine attached to vertebral body 8. As shown, a cuff 10 is positioned on the spinous process 8. The cuff 10 can have a flexible and/or elastomeric body. The cuff 10 has a receiving cavity 10c that receives a length of the spinous process therein. Referring to FIG. 2A, the size of the cuff 10 can vary for different individuals and different spinous processes. Examples of suitable lengths “L” include, but are not limited to, from about 3 mm to about 30 mm, typically between about 8-20 mm. Examples of widths “W” include, but are not limited to, between about 5-30 mm, typically between about 8 mm to about 28 mm. The cuff 10 can have varying thickness over its length or may have a constant thickness. The cuff 10 can have a thickness between about 0.1 mm to about 6 mm, typically between about 1-3 mm. The cuff 10 can be a unitary body or may be formed by one or more attached members. As shown in FIG. 2A, the cuff 10 is a unitary body with its profile (width) narrowing along its length. In position, the narrower portion resides at the most posterior portion of the spinous process.

In some embodiments, the cuff 10 can have an outer surface with a coefficient of friction that allows for ease of motion and/or reduced contact friction (less wear) between adjacent spinous processes. An exemplary coefficient of friction of Salubria® against Salubria® with a moist interface is within the range of between about 0.1 to 0.001, typically about 0.02. Coefficient of friction values for Salubria® against bone can vary depending, for example, on quality of local bone and/or cartilage if present, presence of mesh or not at device interface, etc. . . .

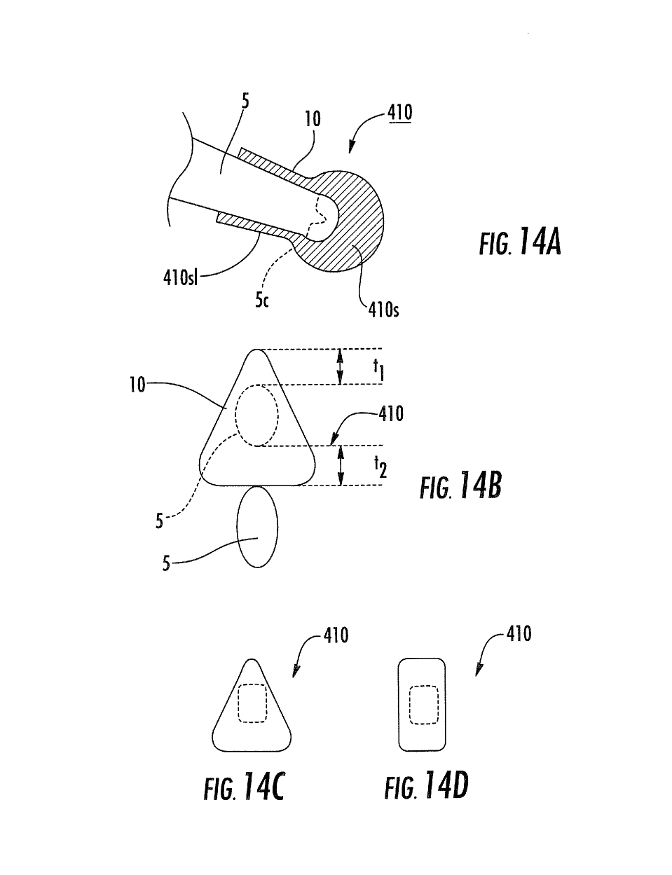

As shown in FIG. 2B, the cuff 10 can include a mesh material 10m that can provide for tissue ingrowth and/or structural reinforcement of the molded body. Exemplary pore sizes for tissue ingrowth and/or bone ingrowth into the exposed mesh scaffold is typically between about 0.5 mm to about 1 mm. For example, a mesh can be molded to the cuff 10 to add an integrated scaffold for tissue ingrowth. FIGS. 2C and 2D illustrate the mesh 10m with an integrated surface molded to the implant body and an exposed surface (or portions thereof) 10e that allows for tissue-ingrowth into the mesh 10m. As shown, the mesh 10m is partially embedded to define a scaffold of partially exposed mesh that resides inside of the cuff. FIG. 2D illustrates that the mesh is integrally attached to the molded body on one primary surface and to a sub-thickness thereof, while all or portions of the other primary surface is exposed 10e to a partial depth mp of the mesh thickness “mₜₕ” and able to allow tissue in-growth. Thus, to facilitate fixation, the partially embedded scaffold, e.g., the partially exposed mesh 10e can be placed in the inside of the cuff, in the superior and/or inferior recesses of the H and U shaped interspinous devices that contact the received spinous process bone. For additional discussion of syStems and methods for providing locally exposed mesh, see, co-pending, co-assigned, U.S. Provisional Application Ser. No. 60/885,682. Fixation members can extend from the scaffold 10m (not shown). The mesh 10m can provide improved attachment of flaps (if required for potential hardware attachment) to the deformable core of the device, where used (see, e.g., FIG. 13A, 320).

Alternatively or additionally, as will be discussed further below, the mesh can define an extension or other attachment and/or fixation member for the device to be affixed to the spinous processes. Also or alternatively, the scaffold (mesh) can be used as reinforcement to the device, which allows more strength and also more flexibility and, where hydrogel materials are used to form the cuff 10, the mesh 10m may allow for use of a reduced hydrogel formulation. The mesh 10m can be any suitable thickness and pore pattern configured to promote tissue in-growth, and may typically be between about 0.5 mm to about 5 mm thick, more typically between about 0.7 mm to about 2 mm thick, such as about 0.75 mm thick. In some embodiments, the mesh 10m can be polyester mesh that may be extruded, knitted, braided, woven or otherwise formed into a mesh pattern. In some embodiments, the mesh 10m comprises a multi-filament fiber(s) that can provide increased strength over conventional polyester material. For example, the mesh 10m can comprise yarns of a polyester mesh multifilament fiber that, for example, can be made out of a High Tenacity Polyester Teraphthalate (HTPET), which typically has a longer molecular chain than conventional polyester material, therefore providing more strength to the mesh than a regular polyester material. In some embodiments, the mesh can be a high strength mesh that using a ball burst test (ref. ASTM D3787-01), can have a burst value between about 1500-3000 N and also a slope of the linear portion of the load/displacement curve of between about 150-300 N/mm. An example of another fabric mesh is DACRON fabric with a thickness that is typically between about 0.25 mm to about 3 mm. One embodiment of DACRON mesh is about 0.7 mm thick, similar to that available as Fablok Mills Mesh #9464 from Fablok Mills, Inc., located in Murray Hill, N.J.

FIG. 3A illustrates that at least one strap or band 12 can be used to secure the cuff 10 to the spinous process 5. The band 12 can be a suture, polymer member or metal member. Typically the at least one band or strap 12 is positioned on an anterior portion of the spinous process (upstream of the irregular shaped posterior bone). FIG. 3B illustrates the cuff 10 with at least one attachment tab 13 that can be used to anchor the cuff 10 to local bone structure. The tab 13 can comprise a material extension that is attached to the cuff body. The material extension can be the same material as that forming the primary cuff body or may be a different material, whether porous or non-porous, having suitable structural properties to allow attachment. In some embodiments, the at least one tab 13 is formed by a mesh material 10m such as a fabric or metallic mesh, such as the mesh described above.

In some embodiments, the at least one tab 13 is defined by mesh (fabric, polymer, and/or metal) being integrally molded or attached to the cuff body. The tab 13 can be reinforced with a coating or laminate structure. The coating can include a polyvinylalcohol (PVA) cryogel. FIG. 4 illustrates that the tab 13 may have a preformed aperture 14, and may have a plurality of apertures, one or more of which can receive a bone anchor 15 (FIG. 3B) such as a staple, suture or bone screw. In other embodiments, the bone anchor 15 can be directly inserted into to the member 13 and bone without preforming apertures 14. In some embodiments, the apertures 14 may be macropores for promoting bone in-growth.

The cuff 10 can be configured as an elastomeric MRI compatible implant. The cuff implant 10 can have a substantially compliant, but sufficiently rigid body so as to be flexible but relatively stiff to provide a desired compressive modulus of elasticity.

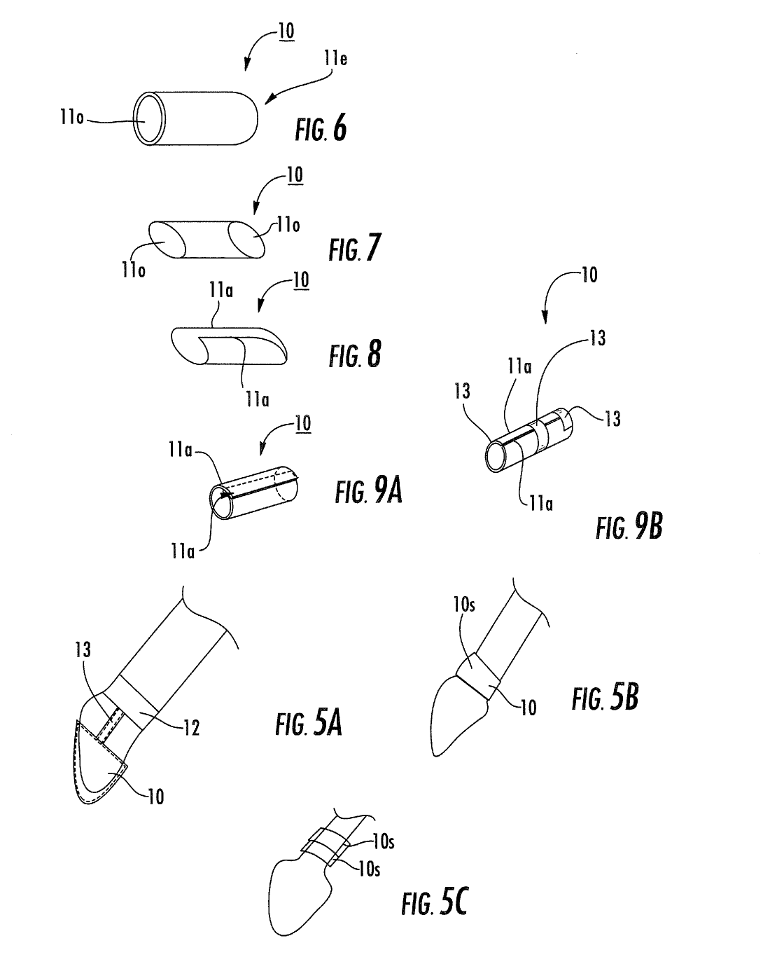

FIG. 5A illustrates that the cuff 10 can reside on a posterior portion of the spinous process and be held in position via at least one axially extending tab extension 13 and a circumferentially extending band 12. FIG. 5B illustrates that the cuff 10 may comprise a single body 10s that is configured to occupy a minor axially inward portion of the spinous process. A plurality of the smaller cuffs 10s can be used on a spinous process as well (not shown), or the smaller cuff 10 can be used with other cuff configurations (not shown). Further, the smaller cuffs 10s can be stacked in partially or completely overlapping fashion over each other. 631 FIGS. 6-8 illustrate exemplary configurations of the cuff 10. As shown in FIG. 6, the cuff 10 has a substantially closed (typically totally enclosed) end portion 11c and an open end portion 11o. FIG. 7 illustrates that the cuff 10 can have two axially opposing open end portions 11o with a closed, typically continuous, perimeter surface therebetween. FIG. 8 illustrates the cuff 10 can have axially extending open long edges 11a that can be closed together, abutting or wrapped over (see FIG. 9A) or spaced apart (FIG. 9B) when in position. The long edges 11a can be held together using straps or bands 12 (FIG. 3A) and/or or by adhesively attaching the long edge portions 11a or tabs 12 in situ or using VELCRO material or a similar attachment configuration. Any one or combinations of these features with tabs, staples, bone anchors, sutures or other structural supports may also be used.

For the embodiments shown in FIGS. 8, 9A and 9B, the cuff body 10 can be a flexible, resilient, sufficiently malleable substantially planar or flat member that can be wrapped about the target spinous process and attached in situ. In other embodiments, the cuff 10 can have a preformed curved shape that is configured to allow the long edges to be pushed closer together in situ. Alternatively, the cuff 10 can be configured to remain in the shape with its long edge portions 11a remaining apart in position (such as in the shape shown in either FIG. 8 or FIG. 9B).

In particular embodiments, at least a portion of the cuff can substantially conform to an underlying bone surface. The cuff 10 can be stretched radially outward and pulled a distance over the posterior portion of the bone to position the cuff 10 in a desired location. A biocompatible lubricant can be applied to the bone and/or the cuff 10 to help slide a snug fit and/or stretchable cuff 10 into location.

In some particular embodiments, the cuff implant 10 can be used without any external tabs (not shown). In some embodiments, where attachment is desired, the device 10 may be self-anchoring, adhesively attached, glued or otherwise anchored to the bone without requiring bone anchors extending outside the bounds of the primary body. For example, the inner surface of the implant 10 can include barbs or anti-migration spikes that extend into bone (not shown). Combinations of the attachment mechanisms described herein can also be used (i.e., barbs and mesh covers or tabs).

The cuff implant 10 can have a solid elastomeric body with compressive strength that is typically less than about 100 MPa (and typically greater than 1 MPa), with an ultimate strength in tension and/or compression that is generally greater than about 100 kPa. The implant 10 can be configured to withstand a compressive load greater than about 1 MPa while allowing motion between the adjacent articulating bones of the facet joint.

The cuff implant 10 can be made from any suitable elastomer capable of providing the desired shape, elasticity, biocompatibility, coefficient of friction, and strength parameters. The cuff implant 10 can be configured with a single, uniform average durometer material and/or may have non-linear elasticity (i.e., it is not constant). The cuff implant 10 may optionally be configured with a plurality of durometers, such as a dual durometer implant. The cuff implant 10 can be configured to be stiffer on a posteriormost portion. In some embodiments, the implant 10 can be configured to have a continuous stiffness variation across its thickness (or length), instead of two distinct durometers. A lower durometer corresponds to a lower stiffness than the higher durometer area. For example, one region may have a compressive modulus of elasticy that is between about 11-100 MPa while the other region may have a compressive modulus of elasticity that is between 1-10 MPa.

The cuff implant 10 can have a tangent modulus of elasticity that is about 1-50 MPa, typically between about 1-10 MPa, typically about 3-5 MPa, and a water content of between about 10-60%, typically about 50%. Suitable compressive Tangential Modulus testing parameters are stated below.
Test type: unconfined compression
Fixtures: flat platens, at least 30 mm diameter
Rate: 25.4 mm/sec to 40% strain
Temperature: room temp (22° C.)
Bath: samples stored in saline or water until immediately before test
Samples: cylinders, 9.8±0.1 mm height, 9.05±0.03 mm diameter
Compressive Tangential Modulus calculated at 15, 20, and 35% strain

In some embodiments, the implants 10 can comprise polyurethane, silicone, hydrogels, collagens, hyalurons, proteins and other synthetic polymers that are configured to have a desired range of elastomeric mechanical properties. Hydrogels and collagens can be made with compressive elasticity values less than 20 MPa and greater than 1.0 MPa. Silicone, polyurethane and some cryogels can be configured to have an ultimate tensile strength greater than about 100 or 200 kiloPascals.

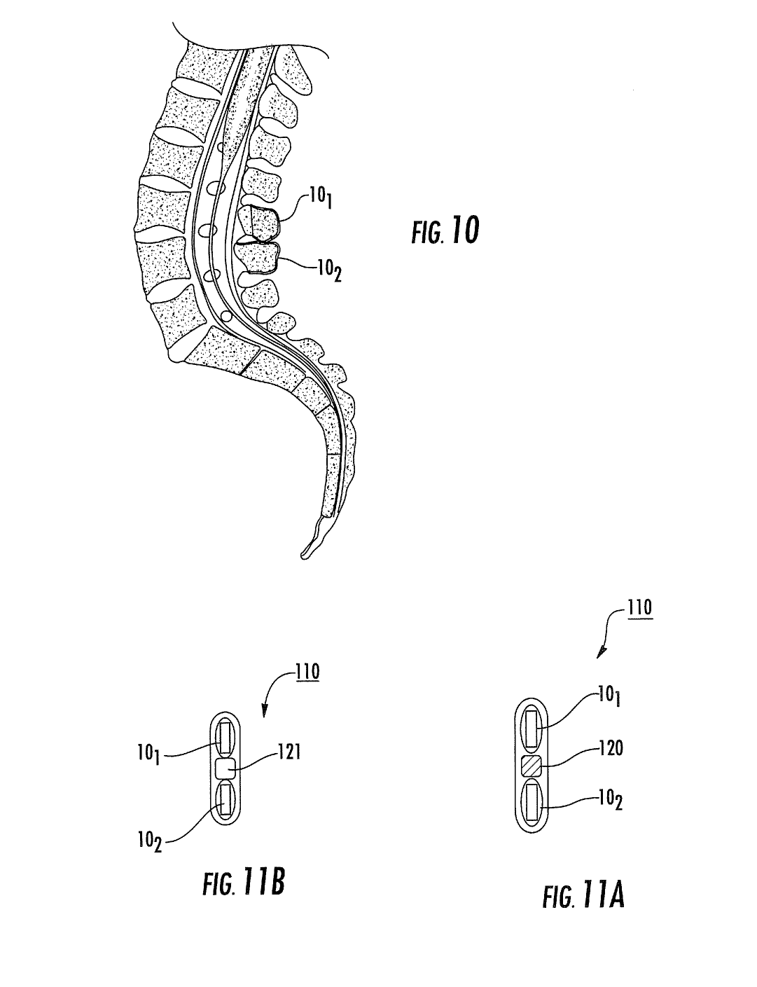

FIG. 10 illustrates that the cuffs 10 can be used in pairs, with first and second cuffs 101, 102, respectively inserted over adjacent spinous processes.

FIG. 11A illustrates a cuff pair 110 with attached cuffs 10₁, 10₂ and a flexible pillow 120 therebetween. The pillow 120 can have increased resiliency relative to the cuff bodies 10₁, 10₂. The pillow 120 may be elastomeric and/or comprise entrapped fluid therein. In other embodiments, the pillow 120 may have the same stiffness or resiliency and/or be stiffer than the cuff bodies 10₁, 10₂. FIG. 11B illustrates a cuff pair 110 with two attached cuffs 10₁, 10₂ and an open gap 121 therebetween. The cuff pairs 110 can be configured to allow motion of the respective spinous process and/or between the two processes. The cuff pair 110 can employ one or more of the features described herein with respect to other embodiments, such as material, material properties, sizes, bone attachment mechanisms, and the like.

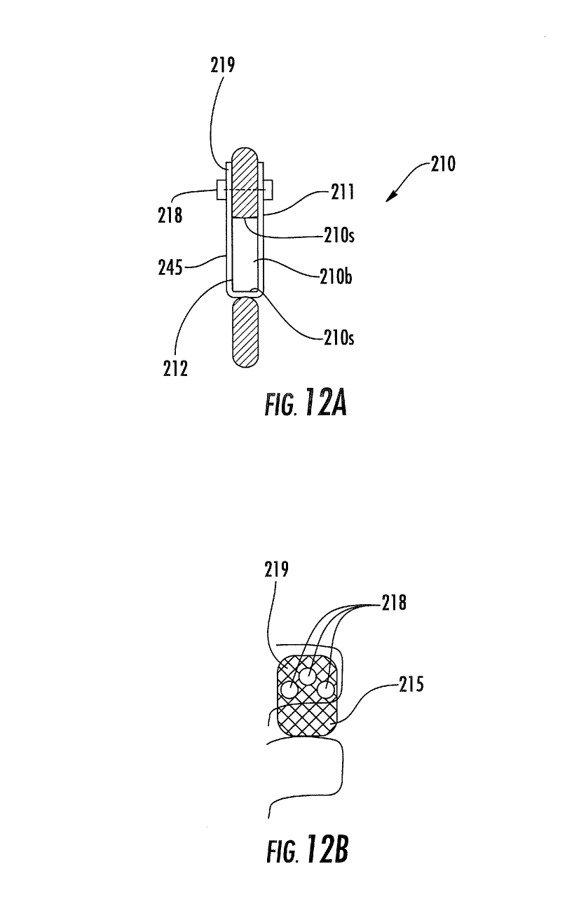

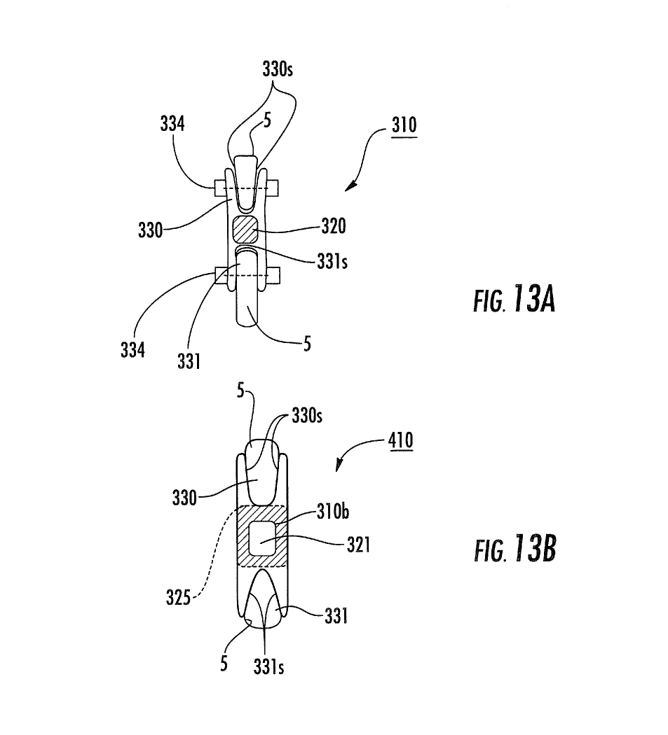

FIGS. 12A and 12B illustrate another embodiment of a spinous process implant 210. In this embodiment, the implant 210 includes an elastomeric body 210b with upwardly extending sides 211, 212. At least one layer of covering material 215 extends over an outer surface of the sides 211, 212 and beyond the bounds of the body 210b to define bone attachment extensions 219. The body 210b can include convex upper and/or lower surfaces 210s.

The covering material 215 may be one unitary layer of material that covers at least a major portion, and typically substantially the entire outer surface of the body 210b. In some embodiments, the material layer 215 comprises mesh that is integrally molded to a molded freeze-thaw crystalline PVA hydrogel body 210b. Alternatively, two separate pieces of material can be used, one each attached to a respective single side of the body 210b, and the lowermost portion of the body 210b may be exposed and contact adjacent bone rather than be covered by the material 218. A bone anchor 218 can be inserted through the covering material 215 to secure to an upper spinous process. In position, the two adjacent spinous processes can still move relative to each other. One or more of the features described herein with respect to other embodiments, such as material, material properties, sizes, bone attachment mechanisms and the like, may be employed with this embodiment. In addition, although described and shown in FIGS. 12A and 12B as attached to an inferior surface of an upper process, the implant 210 can oriented to face and attach to a superior surface of a lower process and the relative locations and orientations of the features thereof modified accordingly.

FIGS. 13A and 13B illustrate yet another embodiment of a spinous process implant 310. In this embodiment, similar to the cuff embodiment shown in FIGS. 11A, 11B, the implant can include an elastomeric pillow 320 (FIG. 13A) or gap space 321 (FIG. 13B). As shown, the implant 310 includes upper and lower receiving channels 330, 331 with sidewalls 330s, 331s that span opposing sides of the bone 5 thereat. Bone anchors 334 can attach the sidewalls to the bone. The pillow 320 and/or the channels 330, 331 can comprise crystalline PVA hydrogel. In some embodiments, the channels 330, 331 can be formed by folding a material layer to define the channel 330, 331 and integrally molding the material layer to the implant body 310. As shown by the shading inside broken lines in FIG. 13B, the body 310b can include an elastomeric medial portion 325 that has increased rigidity with respect to the pillow 320 and/or channels 330, 331. Again, these embodiments may include features described with respect to other embodiments herein.

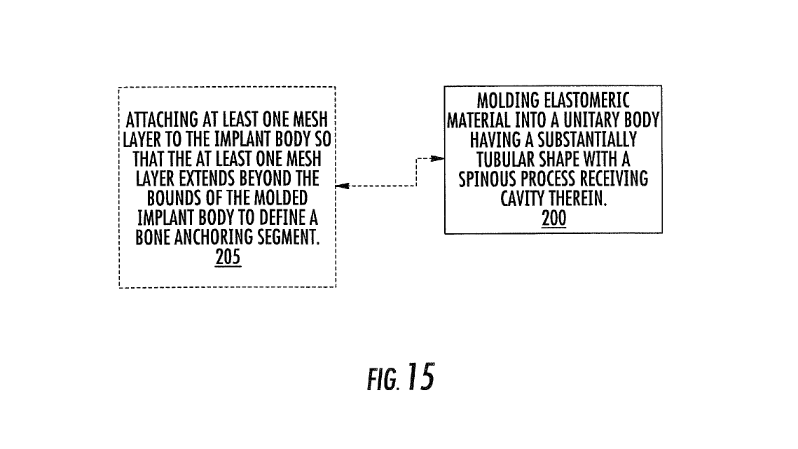

FIG. 14A illustrates another embodiment of a spinous implant 410. As shown, the implant is a cuff 10 with an integral elastomeric and/or flexible spacer 410s. In some embodiments, the spinous process 5 may be surgically cut (designated at 5c) to allow for a desired orientation and spacing in the body. The integral spacer 410 can be bulbous as shown in FIG. 14A with the cuff 10 having increased material at an end portion thereof. As shown in FIG. 14B, the integral spacer 410s may be triangulated with three sides and the lower portion may have a thickness t₂ that is greater than the thickness t₁ of a top portion. In other embodiments, the thickness of the spacer 410s may be substantially the same on top and bottom (and may be configured to define a spacer for each of two adjacent processes). The side thickness may be substantially the same as the top and bottom thicknesses (as shown, for example in FIG. 14C) or the sides may be thinner (not shown). FIG. 14D illustrates that the integral spacer 410s can have a profile that is curvilinear. The spacer 410s can have other profiles, such as substantially hexagonal, rectangular, circular or the like. The posteriormost portion of the spacer 410s can be closed as shown, but can also be optionally (partially or totally) open. The implant 410 can include additional features such as one or more of those described with respect to other embodiments herein.

It is noted that in some embodiments, the cuff 410 can comprise a metal, such as a metal tube, which can merge into an elastomeric spacer 410s. In other embodiments, the cuff 410 can have a unitary body that defines the sleeve as well as the spacer 410s. In yet other embodiments, different elastomeric materials may be used to form different portions of the cuff 410. For example, the spacer 410s may comprise a flexible, resilient elastomeric material with a first stiffness and the sleeve 410s connected thereto can have a lesser or greater stiffness. In some embodiments, the implant 410 body can be formed of a crystalline PVA hydrogel.

The bone may be prepared before placing the implant thereon. For example, the target surface can be gently scraped or made to bleed while maintaining the shape of the bone to promote a bioresponse to facilitate a tissue attachment process.

It is contemplated that in some embodiments the implant 10, 110, 210, 310, 410 can be provided in a range of predetermined sizes to allow a clinician to choose an appropriate size for the patient. That is, any of the implants 10, 110, 210, 310, 410 can be provided in at least two different sizes with substantially the same shape or with different shapes fit to the specific target facet joint/bone. In some embodiments, the implant 10, 110, 210, 310, 410 can be provided in small, medium and large sizes. Further, the sizes can be configured according to the intended implant position, such that some implants have different sizes—e.g., T2, C, and/or L3-L4 implants may have a different size from L4-L5 implants. In some embodiments, an implant can be customized (sized) for each respective patient.

The implants 10, 110, 210, 310, 410 may optionally include one or more radiopaque markers to allow for easier viewing in medical images. The radiopaque marker may include indicia to allow a clinician to see if the center of the implant has migrated over time (shown in broken line as an alignment cross). In other embodiments, the material itself may be configured to be radiopaque.

The implant 10, 110, 210, 310, 410 can be fabricated in any suitable manner, such as, for example, extruded, cut, stamped, and/or molded.

Elastomers useful in the practice of the invention include silicone rubber, polyurethane, polyvinyl alcohol (PVA) hydrogels, polyvinyl pyrrolidone, poly HEMA, HYPAN™ and Salubria® biomaterial. Methods for preparation of these polymers and copolymers are well known to the art. Examples of known processes for fabricating elastomeric cryogel material is described in Pat. Nos. 5,981,826 and 6,231,605, the contents of which are hereby incorporated by reference. See also, Peppas, Poly (vinyl alcohol) hydrogels prepared by freezing—thawing cyclic processing. Polymer, v. 33, pp. 3932-3936 (1992); Shauna R. Stauffer and Nikolaos A. Peppas.

In some embodiments, the implant 10, 110, 210, 310, 410 comprises a solid crystalline hydrogel body, which can be configured to have substantially its final form before implantation. For example, in some embodiments, the weight percentage of (PVA) used to form the implant body 10, 110, 210, 310, 410 and the hydration thereof is such that the body 10, 110, 210, 310, 410 has limited expansion once in position in the body. The implant 10, 110, 210, 310, 410 can be configured to have less than 5% expansion in situ, typically less than 1% expansion in situ, and more typically less than about 0.5% expansion in situ. An exemplary hydrogel suitable for forming a spinous process prosthesis is (highly) hydrolyzed crystalline poly (vinyl alcohol) (PVA). PVA cryogels may be prepared from commercially available PVA material, typically comprising powder, crystals or pellets, by any suitable methods known to those of skill in the art.

In some embodiments, the tabs and/or mesh covering can be molded, ultrasonically welded, staked, brazed, adhesively attached, screwed, nailed or otherwise affixed, attached and/or mounted to the implant 10, 110, 210, 310, 410. In some embodiments, the mesh can comprise non-elastomeric or non-polymer biocompatible materials including malleable metals, metallic mesh and/or non-porous materials, while in other embodiments, the mesh comprises polyester fibers as discussed above. For non-porous materials, the macropores can be arranged to provide for bone-in growth as needed.

FIG. 15 illustrates exemplary operations that can be used to form an implant 10, 110, 210, 310, 410. As shown, elastomeric material can be molded into a unitary body having a substantially tubular shape with a spinous process receiving cavity therein (block 200). Optionally, at least one mesh layer can be attached to the implant body so that the at least one mesh layer extends beyond the bounds of the molded implant body to define a bone anchoring segment (block 205).

In some embodiments, to mold the implant 10, 110, 210, 310, 410, a moldable material comprising an irrigant and/or solvent and between about 20-70%, typically between about 25 to 60% (by weight) PVA powder crystals can be placed in a mold having the desired implant shape. The PVA powder crystals can have a MW of between about 124,000 to about 165,000, with about a 99.3-100% hydrolysis. The irrigant or solvent can be a solution of about 0.9% sodium chloride. The PVA crystals can be placed in the mold before the irrigant (no pre-mixing is required). The mold can be evacuated or otherwise processed to remove air bubbles from the interior cavity. For example, the irrigant can be overfilled such that when the lid is placed on (clamped or secured to) the mold, the excess liquid is forced out, thereby removing air bubbles. In other embodiments, a vacuum can be in fluid communication with the mold cavity to lower the pressure in the chamber and remove the air bubbles. The PVA crystals and irrigant can be mixed once in the mold before and/or after the lid is closed. Alternatively, the mixing can occur naturally without active mechanical action during the heating process.

The irrigant and PVA crystals in the mold are heated. Typically, the mold with the moldable material is heated to a temperature of between about 80° C. to about 200° C. for a time sufficient to form a solid molded body. The temperature of the mold can be measured on an external surface. The mold can be heated to at least about 80-200° C. for at least about 5 minutes, typically between about 10 minutes to 4 hours. The temperature can be measured in several external mold locations. The mold can also be placed in an oven and held in the oven for a desired time sufficient to bring the mold and the moldable material to suitable temperatures. The molds can be held in an oven at about 100-200° C. for about 2-6 hours. The higher range may be needed when several molds are placed therein, but different times and temperatures may be used depending on the heat source, such as the oven, the oven temperature, the configuration of the mold, and the number of items being heated.

In some embodiments, osteoconductive material, such as, for example, calcium salt can be placed on the inner or outer surfaces of the mesh layer and/or the inner mold surfaces to coat and/or impregnate the mesh material to provide osteoconductive, tissue-growth promoting coatings. The mold and mesh can be configured to provide the bone attachment extension segments discussed above.

After heating, the implant body can be cooled passively or actively and/or frozen and thawed a plurality of times until a solid crystalline implant is formed with the desired mechanical properties. The molded implant body can be removed from the mold prior to the freezing and thawing or the freezing and thawing can be carried out with the implant in the mold. Alternatively, some of the freeze and thaw steps (such as about 2-4 cycles) can be carried out while the implant is in the mold, then others (such as between about 5-15 cycles) can be carried out with the implant out of the mold. The implants 10, 110, 210, 310, 410 can be sterilized with sterile heated liquid or with radiation or other sterilization methods, typically after packaging in medical pouch or other suitable container to provide a sterile medical product.

After freezing and thawing, the molded implant 10, 110, 210, 310, 410 can be placed in water or saline (or both or, in some embodiments, neither, during subsequent processing). The device 10,110, 210, 310, 410 can be partially or completely dehydrated for implantation, but is typically in its final form to inhibit passive growth in situ. The implant can be a single solid elastomeric material that is biocompatible by cytotoxicity and sensitivity testing specified by ISO (ISO 10993-5 1999: Biological evaluation of medical devices—Part 5: Tests for in vitro cytotoxicity and ISO 10993-10 2002: Biological Evaluation of medical devices-Part 10: Tests for irritation and delayed-type hypersensitivity.). Additional methods of fabricating implants using moldable material such as hyrdogels are described in co-pending, U.S. Patent Application Nos. identified by Attorney Docket Nos. 9537-7 and 9537-5, with respective co-pending provisional Application Ser. Nos. 60/821,182, and 60/761,902, the contents of which are hereby incorporated by reference as if recited in full herein.

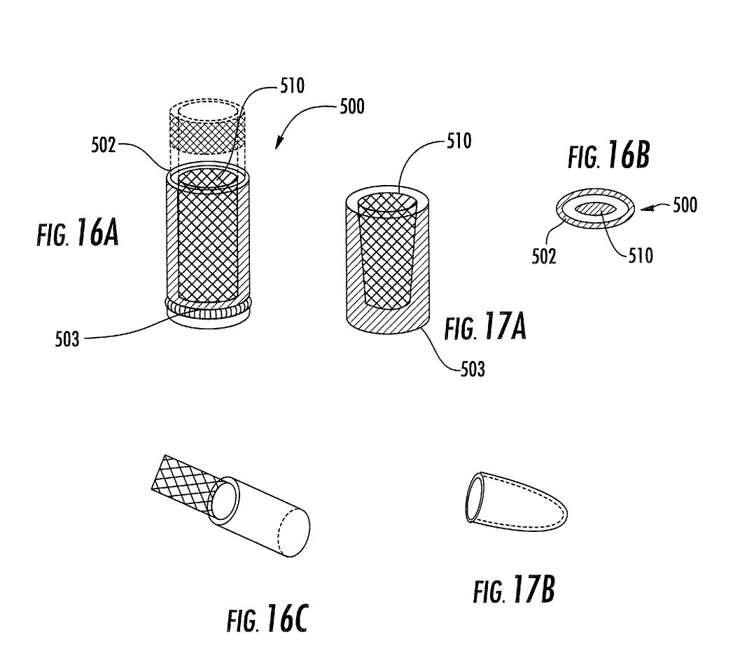

FIG. 16A is a schematic illustration of a primary mold body 500 with an inner post 510 that is spaced apart from a mold wall to define an annulus into which moldable material can be introduced to form a cuff according to particular embodiments of the present invention. The post 510 sits on the floor 503 to prevent moldable material from entering therein, to form a tubular cuff as shown in FIG. 16C. FIG. 17A illustrates the post 510 can reside a distance above the floor 503 to allow moldable material to reside thereunder to allow a tubular closed end cuff to be formed such as that shown in FIG. 17B, or to allow the integral spacer to be formed such as that shown in FIG. 14A. Each mold 500 can be sealably closed using a lid to keep the mold cavity under pressure to mold the implant.

In some embodiments, the mold can be configured with resilient members such as springs (leaf springs or disc springs) inserted underneath one or more screw heads used to attach the mold lid to the mold body. The springs can allow limited or controlled expansion of the mold cavity while keeping the mold closed (retaining the cavity under pressure) to compensate for volume changes as the mold and the molded material therein cool down (the thermal coefficient of the mold and the molded material is typically different). Other thermal compensation mechanisms and configurations may also be used.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein.

That which is claimed:

1. A spinous process implant, comprising:
an elastomeric cuff sized and configured to encase at least a minor portion of a first spinous process.

2. An implant according to claim 1, wherein the elastomeric cuff comprises a crystalline polyvinylalcohol cryogel.

3. An implant according to claim 1, wherein the cuff has a substantially unitary tubular body with one substantially closed end portion and one open end portion.

4. An implant according to claim 1, wherein the cuff has a substantially unitary tubular body with two axially opposing open end portions.

5. An implant according to claim 4, wherein the cuff has long edges that are spaced apart about a portion of an axial length thereof.

6. An implant according to claim 1, wherein the cuff is configured to encase a portion of the spinous process and has a portion that is sized and configured to receive and snugly abut the spinous process that merges into a posterior elastomeric spacer portion.

7. An implant according to claim 6, wherein the cuff substantially conformably contacts the spinous process.

8. An implant according to claim 1, wherein the cuff has a unitary body with a thickness that is between about 0.5 mm to about 5 mm.

9. An implant according to claim 8, wherein the cuff has a length that is between about 5 mm to about 20 mm.

10. An implant according to claim 1, wherein the cuff has a cross-sectional width that is between about 8 mm to about 28 mm.

11. An implant according to claim 1, wherein the cuff has opposing first and second end portions, and wherein the first end portion has a width that is less than that of the second end portion.

12. An implant according to claim 11, further comprising at least one attachment member attached to the cuff sized and configured to secure the cuff in position on the spinous process to thereby inhibit migration.

13. An implant according to claim 1, further comprising a second elastomeric cuff disposed over a neighboring second spinous process whereby the first and second cuffs contact and allow movement between the first and second spinous processes.

14. An implant according to claim 1, further comprising mesh moldably attached to the elastomeric cuff, and wherein the mesh is partially embedded in the molded cuff to define a partially exposed mesh scaffold configured to facilitate tissue in-growth.

15. A method of fabricating a spinous process implant, comprising:
molding elastomeric material into a unitary body having a substantially tubular shape with a spinous process receiving cavity therein.

16. A method according to claim 15, wherein the molding includes forming the substantially tubular shape so that one end is closed and one end is open.

17. A method according to claim 15, wherein the molding includes forming the substantially tubular shape so that both axially opposing ends are open.

18. A method according to claim 15, wherein the molding step comprises using a mold with an elongate center post and a closely spaced sidewall defining an annulus cavity that forms the unitary body shape.

19. A method according to claim 15, wherein the elastomeric material comprises polyvinylalcohol cryogel, the method further comprising integrally molding an attachment material to the unitary body.

20. A method according to claim 15, further comprising molding a mesh material to the elastomeric material whereby the molded mesh defines a mesh scaffold for promoting tissue ingrowth.

21. A spinous process implant, comprising:
a flexible crystalline polyvinylalcohol cryogel body configured to reside between neighboring first and second spinous processes; and
at least one bone attachment member extending from the elastomeric body, configured to attach to the first spinous process to hold the elastomeric body in position, wherein, in position, the implant is configured to allow motion between adjacent spinous process bones.

22. An implant according to claim 21, wherein the flexible body is a unitary thin body of solid crystalline polyvinylalcohol cryogel with at least one layer of mesh material covering at least a major portion of a primary surface thereof and extending outward beyond the body.

23. An implant according to claim 21, wherein the at least one bone extension member comprises a mesh material layer having first and second sides that define a single channel that receives the first spinous process therein, and wherein at least one bone anchor member extends across the channel in a direction that is substantially orthogonal to a direction of the channel sides and serially through the first side of the channel, the spinous process bone, then the second side of the channel.

24. An implant according to claim 23, wherein the mesh material layer substantially covers at least two sides of the flexible body and extends a distance above or below the flexible body.

25. An implant according to claim 21, wherein the at least one bone attachment member comprises a first bone attachment member configured to engage the first spinous process and a second bone attachment member configured to engage the second spinous process.

26. An implant according to claim 25, wherein the first and second bone attachment members comprise a mesh material layer having first and second sides that define a channel that receives a respective one of the first and second spinous process therein, and wherein at least one bone anchor member extends across the channel in a direction that is substantially orthogonal to a direction of the channel sides and serially through the first side of the channel, the spinous process bone, then the second side of the channel.

27. A spinal process implant, comprising:
a first cuff sized and configured to receive at least a minor portion of a first spinous process therein; and
a second cuff attached to the first cuff, the second cuff sized and configured to receive at least a minor portion of a second adjacent spinous process therein, whereby the implant allows motion between the first and second spinous processes.

28. An implant according to claim 27, further comprising an elastomeric cushion member disposed between the first and second cuffs.

29. An implant according to claim 28, further comprising a gap space disposed between the first and second cuffs whereby the gap space resides in a natural channel between the first and second spinous processes.

30. An implant according to claim 28, wherein the cushion comprises crystalline polyvinylalcohol cryogel.

31. An implant according to claim 27, wherein the first and second cuffs comprise crystalline polyvinylalcohol cryogel.

32. An implant according to claim 27, wherein the first and second cuffs comprise a partially embedded mesh scaffold configured to facilitate tissue ingrowth.

33. A medical kit, comprising:
at least one sterilized spinous process cuff enclosed in an aseptic or sterile package.

34. A kit according to claim 33, wherein the cuff comprises an internal mesh scaffold moldably attached thereto to facilitate tissue ingrowth from bone residing therein.

35. A kit according to claim 33, wherein the cuff comprises a sleeve that merges into an integral elastomeric spacer, the cuff configured to encase a posterior portion of the spinous process.

36. A method of treating a spinous process, comprising:
sliding an elastomeric cuff onto a spinous process.

37. A method according to claim 36, further comprising removing a posterior portion of the spinous process before the implanting step.

38. A method according to claim 36, further comprising attaching tabs extending from the cuff to bone.

39. A method according to claim 36, further comprising attaching a strap or band to the cuff.

40. A method according to claim 36, further comprising orienting the cuff so that an exposed mesh scaffold moldably attached to the cuff contacts the spinous process.
